(19) United States
(12) Patent Application Publication
(10) Pub. No.: US 2024/0189067 A1
(43) Pub. Date: Jun. 13, 2024

(54) CLEANING SURGICAL INSTRUMENTS

(71) Applicant: Hedgehog LLC, Howell, MI (US)

(72) Inventors: Curtis Bergquist, Bend, OR (US); Kevin Wittrup, Howell, MI (US); Steven F. Bolling, Ann Arbor, MI (US)

(21) Appl. No.: 18/077,816

(22) Filed: Dec. 8, 2022

(51) Int. Cl. A61B 90/70

(52) U.S. Cl. CPC A61B 90/70; A61L 2202/15

(57) ABSTRACT

A device for cleaning surgical tools includes a housing defining an interior space, the housing comprising a funnel with a narrow end and a wide end, the narrow end of the funnel open to the interior space, a plurality of brushes disposed in the interior space of the housing and aligned with the funnel, a nozzle directed at the plurality of brushes, and a pump operable to dispense a fluid through the nozzle towards the plurality of brushes.

### BACKGROUND

Instruments used during surgery need to be cleaned often to remove debris.

### SUMMARY

Throughout a surgical procedure, surgical instruments are cleaned to remove debris before reintroducing the surgical instruments to the patient. Often, an assistant will clean the surgical instruments, e.g., with water and cloth, between each insertion. The systems and methods described in this specification include a housing containing cleaning components. The cleaning components can include brushes and spraying water, among other components. The housing has an opening through which a surgeon can insert a surgical instrument, and the cleaning components can clean the surgical instrument. These systems can be portable so that the surgeon can position the housing in an easy-to-access location and position.

In an aspect, a device for cleaning surgical tools includes a housing defining an interior space, the housing including a funnel with a narrow end and a wide end, the narrow end of the funnel open to the interior space, a plurality of brushes disposed in the interior space of the housing and aligned with the funnel, a nozzle directed at the plurality of brushes, and a pump operable to dispense a fluid through the nozzle towards the plurality of brushes. Some devices do not include a pump or associated nozzles.

In an aspect, a device for cleaning surgical tools includes a housing defining an interior space, the housing including a first surface opposite a second surface with the first surface inclined at an angle relative to the second surface, the housing further including a funnel with a narrow end and a wide end, the narrow end of the funnel open to the interior space, wherein the funnel extends from the first surface, and cleaning components contained within the housing and aligned with the funnel.

In an aspect, a device for cleaning surgical tools includes a housing defining an interior space, the housing including a funnel with a narrow end and a wide end, the narrow end of the funnel open to the interior space, and a plurality of brushes disposed in the interior space of the housing and aligned with the funnel.

Implementations of these aspects can include one or more of the following features.

In some implementations, the plurality of brushes includes a first brush, a second brush, and a third brush.

In some implementations, the first brush and the second brush are positioned such that an axis of the first brush aligned with an angle between 0 and 5 degrees from an axis of the second brush.

In some implementations, the third brush is positioned such that an axis of the third brush aligned with an angle between 85 and 90 degrees from the axis of the first brush.

In some implementations, the first brush and the second brush are positioned such that an axis of the third brush is aligned with an angle between 0 and 5 degrees from the axis of the first brush.

In some implementations, the third brush is further from the narrow end of the funnel than the first brush and the second brush.

In some implementations, the first brush and the second brush are equidistant from the narrow end of the funnel.

In some implementations, the housing has a first surface opposite a second surface with the first surface inclined at an angle between 0 and 35 degrees relative to the second surface, wherein the funnel extends from the first surface.

In some implementations, the device includes a motor connected to the plurality of brushes, wherein the motor is configured to rotate at least one of the plurality of brushes.

In some implementations, the device includes a filter fluidly connected to the pump.

In some implementations, the device includes a light source mounted on the housing in a position illuminating the funnel.

In some implementations, the second surface is flat and has a surface area in a range of 8 to 15 square inches.

In some implementations, the second surface is attached to a pad that has a larger surface area than the second surface.

In some implementations, the pad is composed of a rigid material.

In some implementations, the light source surrounds the funnel.

In some implementations, the device includes a power button positioned on the first surface.

In some implementations, the device is battery powered.

In some implementations, the cleaning components include a plurality of brushes.

In some implementations, the plurality of brushes are positioned such that axes of each of the plurality of brushes are aligned with an angle between 85 and 90 degrees from an axis of the funnel.

In some implementations, the device includes a light source mounted on the housing in a position illuminating the funnel.

In some implementations, the light source is configured to provide feedback.

In some implementations, the device includes a second funnel.

Surgical instruments can be elaborate, often including curves, recesses, etc., which can make the instruments difficult to clean. The approach described in this specification provides faster, more efficient, and more effective methods and systems for cleaning surgical instruments. The provided methods, systems, and devices allow a surgeon to easily clean surgical instruments, thereby reducing procedure time, reducing interruptions during delicate procedures, and allowing others, e.g., trained assistants, to be available for other tasks.

### DESCRIPTION OF DRAWINGS

FIG. 1 is a perspective view of a cleaning device.

FIG. 2 is a top view of the cleaning device of FIG. 1.

FIG. 3 is an illustration of inner components of the cleaning device of FIG. 1.

FIG. 4 is an illustration of the inner components of the cleaning device of FIG. 1.

FIG. 5 is a perspective view of the inner components of the cleaning device of FIG. 1.

FIG. 6 is a cross-section of the cleaning device of FIG. 1 taken along the A-A plane shown in FIG. 2.

FIG. 7 is a cross-section of the cleaning device of FIG. 1 taken along the B-B plane shown in FIG. 2.

FIG. 8 is a perspective view of the cleaning device of FIG. 1 attached to a pad.

FIG. 9 is a perspective view of another cleaning device.

FIG. 10 is a perspective view of other cleaning components.

### DETAILED DESCRIPTION

This specification describes methods and systems for cleaning surgical instruments. These methods and systems efficiently clean surgical instruments, and provide surgeons with an easy way to clean instruments alone. This can assist a surgeon who is operating solo, or this allows others (e.g., trained assistants) to be available for other tasks.

FIGS. 1-7 illustrate a device 100 that can receive and clean a surgical tool (not shown). The device 100 includes a housing 150 that contains cleaning components, which can be used to clean a surgical tool. For example, the cleaning components can include nozzles that spray water. The cleaning components can also include brushes and other components. The housing 150 has an opening 112 that provides access to the internal cleaning components. A surgeon can insert a surgical instrument (e.g., forceps, a scalpel, etc.) through the opening 112 so that the cleaning components can clean the surgical instrument. The device 100 can be portable so that the surgeon can position the housing in an easy-to-access location and position.

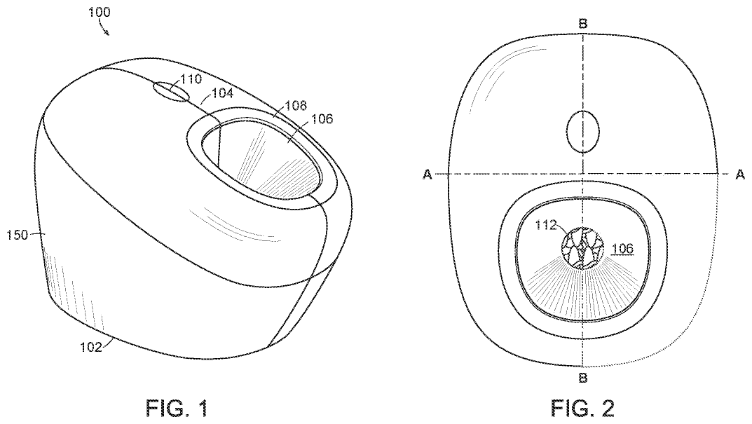

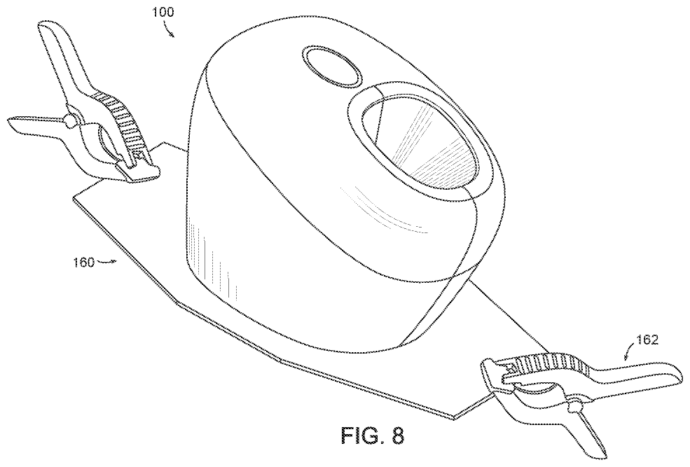

FIGS. 1 and 2 illustrate the external configuration of the device 100. The device 100 has a bottom surface 102 on which the device 100 stands. The bottom surface is configured to support the device in a stable position, for example, on a patient's torso or an instrument tray. In a prototype device, the bottom surface was flat as illustrated in FIG. 7. Some devices have bottom surfaces with other configurations (e.g., with protrusions extending outward from an otherwise planar surface). The bottom surface 102 can be attached to other objects (e.g., through adhesives, fasteners, etc.) to secure the surface 102 to the object. For example, the bottom surface 102 can be attached to surgical drapes and placed on top of a patient's torso. Placing the device 100 on top of the patient during a procedure is advantageous because a surgeon can easily reach the device 100 to clean the surgical instruments that are being used.

In some implementations, the bottom surface 102 includes or attaches to a pad (not shown) that is larger than the device to provide a larger footprint and stabilize the device 100. For example, FIG. 8 illustrates the device 100 attached to a pad 160. The pad can be composed of rigid or flexible material, or a combination thereof. The pad can be clamped to other surfaces or objects, e.g., to surgical drapes, which can be advantageous for the reasons listed above. For example, surgical clamps may be readily available in the surgical environment. Attachments other than clamps can also be used. In some implementations, the pad 160 includes an adhesive that allows the device 100 to be affixed without requiring a clamp. In some implementations, the clamps 162 are positioned directly on the bottom surface 102 of the device 100, and the pad 160 is omitted. The device 100 can be easily portable and disposable. In some implementations, the device 100 is reusable.

The device 100 is lightweight (e.g., under 1.5 lbs) and sized to rest on a patient's torso (e.g., the surface area of the bottom surface can be between about 8 to about 15 square inches). For example, the prototype was 4 inches in length, 3 inches in width, and 3 inches in height. The device 100 has a top surface 104 opposite the bottom surface 102. As shown in FIG. 7, the top surface 104 is inclined at angle α relative to the bottom 102 surface. In the prototype, the top surface 104 was inclined at 20 degrees, but in other implementations the top surface 104 is inclined at other angles. For example, the top surface can be inclined at angles in a range of zero to 35 degrees.

A funnel 106 is seated in the top surface 104 and can receive surgical instruments for cleaning. As discussed above, the top surface 104 provides an ergonomic angle for a surgeon to easily insert a surgical instrument into the device 100 with minor movements and without the instrument leaving the surgeon's hand. For example, the surgeon may be able to insert the surgical instrument into the funnel 106 without having to twist his or her arm in different directions. The funnel 106 directs the surgical instrument into the housing 150, e.g., so that the surgeon does not have to be especially precise during insertion of the surgical instrument.

Some of the cleaning devices include a light source that illuminates the funnel. In the device 100, the funnel 106 is surrounded by a light source 108 (e.g., an LED) so that the surgeon can easily see the entrance of the funnel 106. In some implementations, the light source only partially surrounds the funnel 106. In some implementations, the device 100 does not include a light source 108. For example, the device may not have a light source 108 if it is designed to be used in a well-lit environment.

Some cleaning devices also include a power button to control the device. In the device 100, a power button 110 is positioned on the top surface 104. The power button is connected to a controller. Pressing the power button 110 can turn electronics within the device 100 (e.g., the light source 108, internal electronics, cleaning components, etc.) on or off as desired. In some implementations, pressing the power button 110 multiple times can change the speed and/or power of components within the device 100. In some implementations, the power button enables electronics in the device that detect when an instrument is being presented to the device such that the cleaning components can be activated to clean the instrument and subsequently deactivated when the instrument is withdrawn.

FIG. 2 is a top view of the device 100. The funnel 106 has an opening 112 for surgical instruments to be inserted into the device 100. The funnel 106 directs the surgical instrument into the opening 112 and into the internal cleaning components. The cleaning components (e.g., brushes, nozzles, etc.) clean the surgical instruments, as discussed further below, to remove debris and prepare the surgical instruments to be reintroduced to the patient.

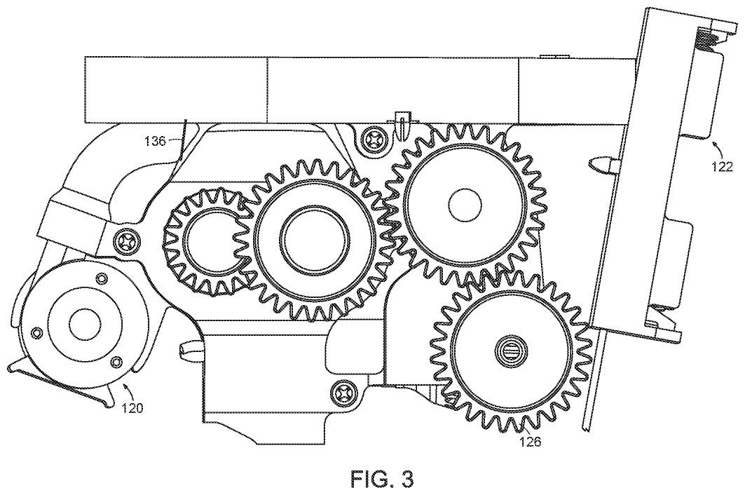

FIG. 3 shows internal components of the device 100. The internal components include a pump 120, which pumps fluid (e.g., water, disinfectant, saline, etc.) into a nozzle to spray surgical instruments as part of the cleaning process. Some devices are configured to clean the fluid so it can be reused. For example, the device 100 includes a filter 136 in its fluid circuit. In some implementations, the filter 136 separates two fluid chambers to prevent debris from being recirculated to the inlet of the pump 120.

In some implementations, the used fluid is separated from the fluid source such that used fluid is not reused. The fluid can contain an agent (e.g., a luminescent agent and/or dying agent) that adheres to biological material. The agent can be optically detectable (e.g., in the presence of visible light, ultraviolet light, etc.) so that a surgeon can determine whether biological material remains on the surgical instrument before reintroducing the surgical instrument to the patient.

Some devices do not include a pump or the associated nozzles. These devices typically include the other components of the device 100 (e.g., brushes and motor) but have reservoir that the brushes are positioned within. The reservoir can be filled before use during a procedure (e.g., providing a water or cleaning solution level above the brushes). Rotation of the brush or brushes circulates the water/cleaning fluid within the reservoir. In use, as an instrument being cleaned is inserted into the device, the instrument submerges into the water/cleaning solution and engages with the brushes. The fluid can help loosen materials attached to the instrument as well as carrying removed materials away from the instrument.

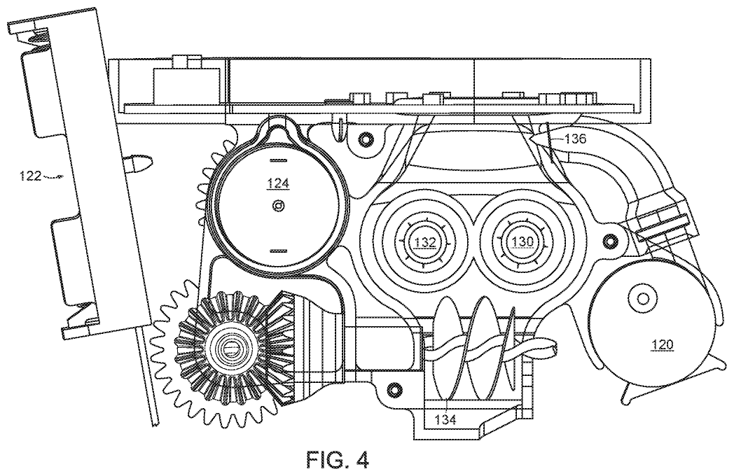

The device 100 includes a power source 122. Although depicted as a battery pack, other power sources (e.g., plugs) can be used. The power source 122 provides power to the pump 120, the light source 108, and a motor 124 (shown in FIG. 4) that drives a set of gears 126 connected to brushes. The set of gears 126 is designed to rotate the brushes at speeds that vary from the motor speed, e.g., through gear reduction. In some implementations, the motor 124 includes internal gears such that the brush rotation speed is significantly less than the speed of the ungeared electric motor (e.g., less than a 50:1 ratio).

In some implementations, the brushes rotate within a range of about 40 rpm to about 200 rpm. The speed of the brushes can impact both the cleaning process (e.g., the amount of time an instrument should be inserted within the device 100) and the noise created by the device 100. In some implementations, the speed of the motor and/or the speed of the brushes is kept at a low rpm (e.g., 100 rpm) to maintain a low sound level, allowing surgeons to concentrate on the surgery being performed.

FIG. 4 illustrates a side view of the internal components of the device 100 taken from the opposite side than FIG. 3. In FIG. 4, some of the components are illustrated as partially transparent for clarity of the overall illustration.

Three brushes 130, 132, 134 are connected to the gears 126 such that they each rotate when the motor 124 is powered on. Two of the brushes 130, 132 are parallel (e.g., such that an axis of the first brush aligned with an angle between 0 and 5 degrees from an axis of the second brush), and the third brush 134 is perpendicular to the parallel brushes 130, 132 (e.g., such that an axis of the third brush aligned with an angle between 85 and 90 degrees from to the axis of the first brush). The brushes 130, 132 are equidistant from the opening, and the third brush 134 is further from the opening than the other two brushes 130, 132. This configuration of brushes extends around an inserted surgical instrument and increases the likelihood that the surgical instrument will come into contact with at least one of the brushes 130, 132, 134. The geometry of the housing 150 functions to help guide an instrument to a brush. Having the brushes extending around the surgical instrument can be helpful, e.g., because surgical instruments can be elaborate, often including curves, recesses, etc. In some implementations, the parallel brushes 130, 132 are touching to force the surgical instrument into contact with at least one of those brushes. The brushes can physically dislodge debris from the instrument when the instrument comes into contact with the brushes. The third brush 134 can be spaced apart from the top two brushes, as illustrated. In some implementations, the third brush 134 is touching the other two brushes 130 132. The distance between the brushes 130, 132, 134 can be designed for different surgical tools (e.g., forceps, scissors, probes, tweezers, etc.). In some implementations, the brushes 130, 132, 134 are identical. In the prototype, each of the brushes was a helical brush. In other implementations, the brushes 130, 132, 134 can be other types of brushes and can be different from each other.

A nozzle 136 is connected to the pump 120 to spray surgical instruments with fluid as part of the cleaning process, as described above. In the prototype, the nozzle was pointed laterally. In other implementations, the nozzle is pointed in other directions. For example, in some implementations the nozzle sprays the brushes and instrument from underneath the brushes. The nozzle 136 can spray the surgical instrument with varying levels of pressure: in some implementations, the nozzle 136 is low pressure and serves to simply wet the instrument and brushes. In other implementations, the nozzle 136 is high pressure and serves to remove debris from the surgical instrument. The nozzle 136 can also rinse the surgical instrument and brushes to flush debris. As previously noted, some devices do not include a pump or associated nozzles.

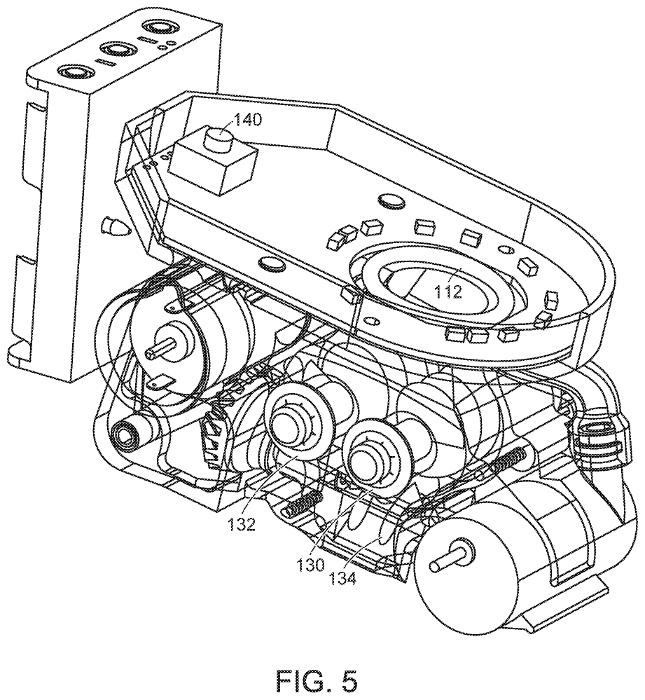

FIG. 5 is a perspective view of the inner components of the device 100. The opening 112 of the funnel 106 directs inserted surgical instruments into the center of the brushes 130, 132, 134. The nozzle 136 also sprays fluid at the center of the brushes 130, 132, 134. Directing the surgical instrument into the center of the brushes and into the spray cleans the instrument efficiently, e.g., by bringing the surgical instrument into contact multiple components within the device 100. A switch 140 is connected to the power button 110, such that pressing the power button 110 can turn the electronics (e.g., the motor 124, the light source 108, the pump 120, etc.). The switch 140 is a mechanical switch, but other types of switches (e.g., magnetic switches) can be used.

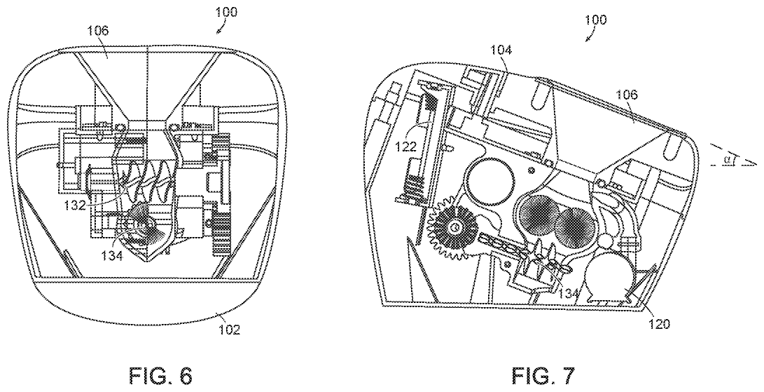

FIG. 6 is a cross-sectional view of the device 100 along the A-A plane shown in FIG. 2. The cross-section provides a detailed view of the inner components. This illustration highlights how the inner components are positioned within the device 100 so that the opening 112 of the funnel 106 is centered over the cleaning components and directs inserted surgical instruments into the center of the brushes 130, 132, 134.

FIG. 7 is a cross-sectional view of the device 100 along the B-B plane shown in FIG. 2. This cross-section provides another detailed view of how the inner components are positioned within the device 100. The components are generally inclined along with the inclined surface 104, and the funnel 106 is generally perpendicular to the brushes 130, 132, 134 (e.g., positioned such that axes of each of the brushes are aligned with an angle between 85 and 90 degrees from an axis of the funnel). The funnel 106 is perpendicular to the brushes so that surgical instruments are inserted generally perpendicularly to the brushes 130, 132, 134. Inserting the surgical instrument perpendicularly provides surface contact between instrument and the three brushes to sufficiently clean the instrument. Inserting the instrument perpendicularly also provides approximately equal contact with each of the three brushes.

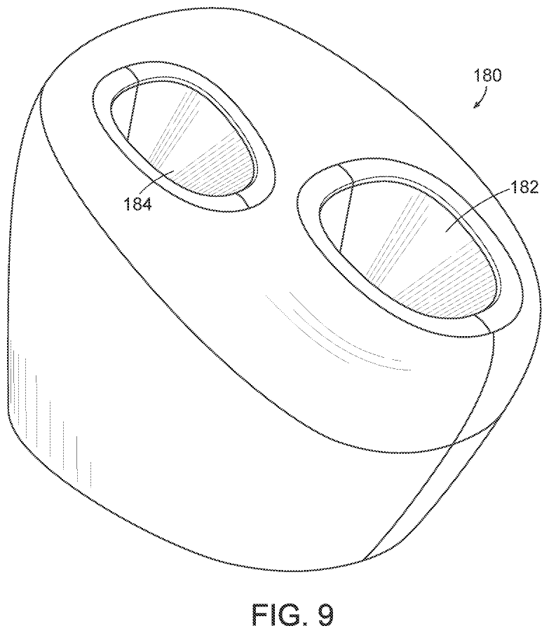

Although the device is described as having a single funnel that introduces the surgical instrument to all of the cleaning components, in some implementations the device has multiple funnels that each introduce the surgical instrument to individual components. For example, FIG. 9 illustrates a cleaning device 180 having two funnels 182, 184. Each of the funnels 182, 184 have a narrow end open to the interior of the device 180. The first funnel 182 introduces the surgical instrument to the brushes, and the second funnel 184 introduces the surgical instrument to the nozzle for rinsing. When cleaning an instrument, a surgeon can insert the instrument into the first funnel 182 to be brushed until it is free of debris. Then, the surgeon can insert the instrument into the second funnel 184 to be rinsed by the nozzle. In some implementations, the separate funnels are configured to accommodate specialized instruments.

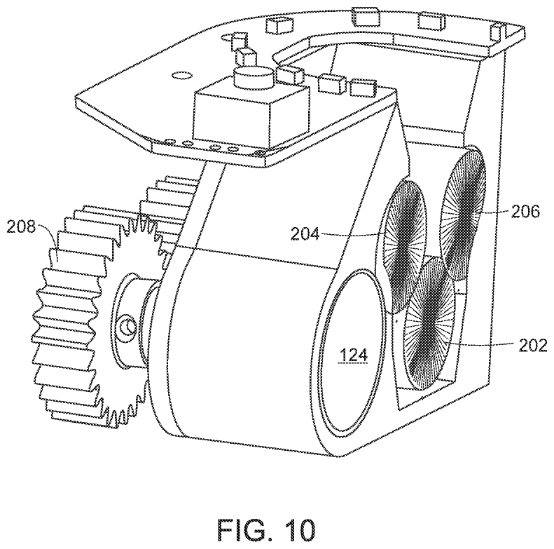

In some implementations, cleaning components are aligned differently within the device. For example, FIG. 10 illustrates an alternative configuration of cleaning components. The configuration shown in FIG. 10 can be contained within a housing similar to the housing 150 of FIG. 1. The motor 124 can be the same as the motor described above. One brush 202 is connected to gears 208 such that it rotates when the motor 124 is powered on. Two of the brushes 204, 206 are static and do not rotate. All three of the brushes are parallel (e.g., an axis of the first brush is aligned with an angle between 0 and 5 degrees from an axis of the second brush and aligned with an angle between 0 and 5 degrees from an axis of the third brush), The two brushes 204, 206 that are static are equidistant from the opening, and the third brush 202 is further from the opening than the other two brushes 204, 206. This configuration of brushes extends around an inserted surgical instrument and increases the likelihood that the surgical instrument will come into contact with at least one of the brushes 202, 204, 206. The geometry of the housing functions to help guide an instrument to a brush. Surrounding the surgical instrument can be helpful, e.g., because surgical instruments can be elaborate, often including curves, recesses, etc. In some implementations, the brushes 204, 206, 208 are touching to force the surgical instrument into contact with at least one of those brushes. The brushes can physically dislodge debris from the instrument when the instrument comes into contact with the brushes. The distance between the brushes 202, 204, 206 can be designed for different surgical tools (e.g., forceps, scissors, probes, tweezers, etc.). In some implementations, the brushes 202, 206, 208 are identical. For example, each of the brushes can be a helical brush. In other implementations, the brushes 202, 204, 206 can be other types of brushes and can be different from each other. In some implementations, two brushes rotate and a third brush is static. In some implementations, all of the brushes rotate. In other implementations, none of the brushes rotate. In some implementations, the brush 204 rotates while the other two brushes 202, 206 are static. In other implementations, the brush 206 rotates while the other two brushes 202, 204 are static. Implementations can include more or fewer brushes, each of which can rotate or be static.

Although the device is described as having brushes, other cleaning components can be used alternatively or in addition to brushes. For example, sponges, bristles, etc. can be used to clean surgical instruments. In some implementations, one or more cleaning components may be fixed (i.e., non-rotating). In some implementations, a high pressure fluid (or gas) is used to clean the surgical instruments, and no brushes contact the surgical instruments.

In some implementations, the device includes electronic sensors (e.g., motion sensors, metal detectors, etc.) that detect the presence of a surgical instrument within the funnel and/or within the opening. The sensors enable the electronics to activate the cleaning components only when an instrument is detected, thereby reducing energy consumption and noise generated by the device. This can also allow for an automated device without a power button, i.e., the device will power on and off automatically.

In some implementations, the device includes optical sensors within the funnel or near the funnel such that the optical sensors can detect the presence of biological material or debris on the surgical instrument after cleaning. For example, in some implementations where the fluid contains an agent (e.g., a luminescent agent and/or dying agent) that adheres to biological material, the optical sensors are configured to detect the agent. A processor can analyze the signals received from the optical sensors and determine the presence and/or amount of biological materials. For example, the processor can evaluate the degree to which biological material is remaining on the device. In some implementations, the device can give visual and/or auditory feedback (e.g., via the light source and/or via a speaker) to the surgeon regarding the cleanliness of the surgical instrument.

This specification describes devices, methods, and systems for cleaning surgical instruments. It will be appreciated that various changes may be made by those skilled in the art without departing from the spirit and scope of this disclosure.

What is claimed is:

1. A device for cleaning surgical tools, the device comprising:
a housing defining an interior space, the housing comprising a funnel with a narrow end and a wide end, the narrow end of the funnel open to the interior space;
a plurality of brushes disposed in the interior space of the housing and aligned with the funnel;
a nozzle directed at the plurality of brushes; and
a pump operable to dispense a fluid through the nozzle towards the plurality of brushes.

2. The device of claim 1, wherein the plurality of brushes comprises a first brush, a second brush, and a third brush.

3. The device of claim 2, wherein the first brush and the second brush are positioned such that an axis of the first brush aligned with an angle between 0 and 5 degrees from an axis of the second brush.

4. The device of claim 3, wherein the third brush is positioned such that an axis of the third brush aligned with an angle between 85 and 90 degrees from the axis of the first brush.

5. The device of claim 4, wherein the third brush is further from the narrow end of the funnel than the first brush and the second brush.

6. The device of claim 5, wherein the first brush and the second brush are equidistant from the narrow end of the funnel.

7. The device of claim 1, wherein the housing has a first surface opposite a second surface with the first surface inclined at an angle between 0 and 35 degrees relative to the second surface, wherein the funnel extends from the first surface.

8. The device of claim 7, wherein the device further comprises a motor connected to the plurality of brushes, wherein the motor is configured to rotate at least one of the plurality of brushes.

9. The device of claim 1, further comprising a filter fluidly connected to the pump.

10. The device of claim 1, further comprising a light source mounted on the housing in a position illuminating the funnel.

11. A device for cleaning surgical tools, the device comprising:
a housing defining an interior space, the housing comprising a first surface opposite a second surface with the first surface inclined at an angle relative to the second surface, the housing further comprising a funnel with a narrow end and a wide end, the narrow end of the funnel open to the interior space, wherein the funnel extends from the first surface; and
cleaning components contained within the housing and aligned with the funnel.

12. The device of claim 11, wherein the second surface is flat and has a surface area in a range of 8 to 15 square inches.

13. The device of claim 12, wherein the second surface is attached to a pad that has a larger surface area than the second surface.

14. The device of claim 13, wherein the pad is composed of a flexible material that conforms to a patient's body.

15. The device of claim 14, further comprising a power button positioned on the first surface.

16. The device of claim 15, wherein the device is battery powered.

17. The device of claim 16, wherein the cleaning components comprise a plurality of brushes.

18. The device of claim 17, wherein the plurality of brushes are positioned such that axes of each of the plurality of brushes are aligned with an angle between 85 and 90 degrees from an axis of the funnel.

19. The device of claim 17, further comprising a pump operable to circulate a fluid past the plurality of brushes.

20. The device of claim 11, further comprising a light source mounted on the housing in a position illuminating the funnel.

21. The device of claim 20, wherein the light source is configured to provide feedback.

22. The device of claim 11, further comprising a second funnel.

23. A device for cleaning surgical tools, the device comprising:
a housing defining an interior space, the housing comprising a funnel with a narrow end and a wide end, the narrow end of the funnel open to the interior space; and
a plurality of brushes disposed in the interior space of the housing and aligned with the funnel.

24. The device of claim 23, wherein the plurality of brushes comprises a first brush, a second brush, and a third brush.

25. The device of claim 24, wherein the first brush and the second brush are positioned such that an axis of the first brush aligned with an angle between 0 and 5 degrees from an axis of the second brush.

26. The device of claim 25, wherein the third brush is positioned such that an axis of the third brush aligned with an angle between 85 and 90 degrees from the axis of the first brush.

27. The device of claim 26, wherein the third brush is further from the narrow end of the funnel than the first brush and the second brush.

28. The device of claim 27, wherein the first brush and the second brush are equidistant from the narrow end of the funnel.

29. The device of claim 23, further comprising a pump operable to circulate a fluid past the plurality of brushes.
